(19) United States
(12) Patent Application Publication
(10) Pub. No.: US 2003/0008306 A1
(43) Pub. Date: Jan. 9, 2003

(54) Circular polynucleotide templates and methods for oligonucleotide synthesis

(76) Inventors: Kenneth Turnbull, Kansas City, MO (US); Janardhanam Selvasekaran, St. Louis, MO (US)

Correspondence Address: HEAD, JOHNSON & KACHIGIAN, 228 W 17TH PLACE, TULSA, OK, 74119, US

(21) Appl. No.: 10/085,395

(22) Filed: Feb. 28, 2002

Related U.S. Application Data
(63) Continuation-in-part of application No. 09/283,569, filed on Mar. 31, 1999.
(60) Provisional application No. 60/080,198, filed on Mar. 31, 1998.

(51) Int. Cl. C12Q 1/68; C07H 21/04; C12P 19/34; C07K 9/00

(52) U.S. Cl. 435/6; 435/91.2; 536/24.3; 530/322

(57) ABSTRACT

A method is provided for forming oligonucleotides by using a circularized polynucleotide template. The circular template utilizes Hoogsten hydrogen bonding to form a triplex with the substrate nucleotides. This overcomes stacking forces and provides for rapid and accurate oligimerization reactions. The circular template may be modified with primers that may be covalently bound.

### BACKGROUND OF THE INVENTION

1. Field of the Invention

The present invention relates to a cost efficient process for large scale synthesis of homopurine digonucleotides (ON's).

2. Prior Art

The completion of the human genome sequencing project has placed an emphasis on methods which will allow functional analysis of the human genome, and prepare the way for diagnostic and therapeutic approaches for disease analysis and treatment. One of the most direct means to mediate protein production and genetic transformation is through antisense and antigene approaches. One of the key limitations which will need to be addressed in bringing this technology to diagnostic and therapeutic application is the ability to produce large scale (gram to kilogram) quantities of desired natural and non-natural oligonucleotides (ON's). There have been no “green processes” reported which address this need in a cost effective manner.

The state-of-the-art techniques for oligonucleotide synthesis using well established, automated, solid-phase chemistry are based on elegant but complex phosphoramidite, phosphite-triester, or H-phosphonate approaches. Recent advances in solid-phase synthesizers allow multi-gram scale (up to 5 mmol) production of pure ON's. Pharmacia's OligoProcess synthesizer is capable of producing kilogram quantities of pure phosphorothioate ON's for clinical trials. Pharmacia's instruments have made extensive progress in overcoming traditional drawbacks of solid support synthesis such as limited reaction rate and yield due to limited permeability and steric hindrance of the heterogeneous reaction mixture. But of far greater concern is the high cost and the environmental impact of high volume waste that is generated by large scale, multistep synthesis of oligomers by solid support approaches. The use of specially synthesized supports, multiple protecting groups, specialized activated derivatives and reagents for couplings and oxidations, the requirement for anhydrous conditions, repeated capping of unreacted groups, and multiple washing cycles results in a high cost for reagents, operation, maintenance and waste disposal. The overall economic and environmental impact is therefore less attractive than a “green” process that would allow the use of cheap starting materials, few reagents, aqueous based chemistry, produce little waste, and allow recycling of unaltered starting materials.

An alternative to solid support chemistry has been the development of solution based methods for large scale ON synthesis under homogeneous conditions. The most attractive approaches incorporate the advantages of solid supports by performing the synthesis on a high molecular weight polymer for ease of purification steps through size exclusion methods. However, the polymer is soluble to maintain reaction homogeneity so that reaction efficiency is high and large scale reactions can theoretically be achieved. The largest scale ON synthesis using this approach has been up to hundreds of milligrams. Although larger scale reactions can theoretically be performed by these solution based approaches, the economic disadvantages of starting material and reagent costs, complex protecting group requirements and high volume solvent use and waste disposal make these approaches as environmentally and economically unattractive as the solid support methods.

An additional approach for ON synthesis is through enzymatic oligomerizations. This approach is appealing in terms of avoiding the costly starting materials and the waste disposal problems, but the potential for large scale ON synthesis is severely limited by several factors. The overexpression of enzymes is a tedious and expensive multistep process which requires time and complicated purification strategies. If enough enzyme could be produced to accomplish kilogram scale ON synthesis, the expense would likely prove too prohibitive. In addition, while the enzymatic oligomerization reaction itself is efficient, the purification of the desired product is again a multistep, laborious and expensive process. An additional limitation to enzymatic approaches is the inability to produce modified ON's. Only naturally occurring ON's can be synthesized enzymatically. The use of ON's for diagnostic and therapeutic applications requires modified, non-natural derivatives in order to afford biodelivery and biostability characteristics to the ON's. The economic prohibitions and limitation to natural ON's by an enzymatic based approach make it unattractive for large scale ON synthesis of biomedicinal utility.

A highly attractive approach to ON synthesis is through non-enzymatic, template directed ligations and oligomerizations. The ability to non-enzymatically direct phosphodiester bond formation of two oligonucleotides in aqueous solution through the action of a phosphate activating reagent and a nucleic acid template was first realized in 1966. Since that time numerous oligonucleotide ligation reactions have been reported in duplex directed systems with single strand DNA templates, where Watson-Crick hydrogen bonding affords the substrate-template association. ON ligations have also been reported in triplex directed systems with double strand templates, where Hoogsteen hydrogen bonding of homopyrimidine ligation substrates to the homopurine strand of a homopryrimidine-homopurine Watson-Crick duplex affords the substrate-template complex. Non-enzymatic, template directed ligation strategies are particularly advantageous for constructing non-natural, modified oligonucleotides. This includes the synthesis of small, circular DNA through the template directed circularization of linear ON's.

Chemically activated, template directed ligation and oligomerization reactions have gained interest for their potential role in prebiotic DNA and RNA synthesis. This area of research has contributed the most significant progress in regard to product turnover for a more catalytic use of the templates. However, application to large scale ON synthesis has not been an addressed objective. While elegant systems have been developed to study template directed oligomerization, the low yield of oligomerization reactions and requirement for activated nucleotide monomers which suffer from hydrolytic degradation and side reactions limit the synthetic utility of existing approaches for large scale ON synthesis.

The higher association of short ON's with DNA templates has resulted in numerous reports of template directed ligation reactions of short ODN's as a less challenging alternative to template directed mononucleotide oligomerizations. Yields as high as 85% have been reported for triplex template directed ligation reactions, although limited to the ligation of longer ON substrates (two 12-mers to afford a 24-mer, GC content=50%).

A largely unexploited potential for template directed oligonucleotide synthesis has been in the development of large scale (gram to kilogram) production of ON's. This potential has likely gone untapped due to the poor turnover rate resulting in inefficient template utilization. One unique approach to this problem was the “rolling” circle DNA synthesis where a single strand, circular DNA template has been used for the enzymatic synthesis of extremely long, single strand DNA products composed of multiple copies of the circular template sequence. While this approach has the potential for large scale ON synthesis, it will be limited by the need for polymerases and restriction enzymes and the limitations of natural ON synthesis.

One of the difficulties experienced by the DNA templates described above when used for oligonucleotide formation is the fact that a significant amount of the energy that facilitates double stranded DNA comes not from complimentary base pair hydrogen bonds, but from the stacking energy acquired from the double helix geometry. DNA polymerase enzymes generally twist the template strand of DNA to eliminate these stacking forces when pairing complimentary base pairs. In order for a DNA template used to synthesize oligonucleotides by means of an organic reaction, it would be necessary to have base specific hydrogen bonds whose cumulative energy value far exceeded that of the stacking energy associated with polynucleotides.

It is therefore desirable to provide a template for the organic non-enzymatic, synthesis of oligonucleotides.

It is also desirable to provide a method of producing relatively large quantities of oligonucleotides in an environmentally friendly manner.

It is also desirable to provide a method for producing relatively large quantities of oligonucleotides having relatively little cost and relatively simple purification steps.

### BRIEF SUMMARY OF THE INVENTION

The present invention provides a new, cost efficient “green” process for the large scale synthesis of homopurine oligonucleotides (ON's). The therapeutic and diagnostic applications of homopurine ON's for antisense and antigene approaches makes large scale ON synthesis (gram to kilogram quantities) of primary importance. The present invention far exceeds all present means for synthesizing ON's in regard to production scale, cost, efficiency and environmental impact. This is accomplished here by the simplicity of the required starting materials and reagents and the aqueous based nature of the reactions. The present invention allows large scale synthesis of modified DNA and RNA oligonucleotides which are required for biodelivery and biostability in diagnostic or therapeutic applications. This methodology also allows extension to oligomerization processes incorporating non-natural nucleotide bases.

The general method involves the use of a polynucleotide template directed reaction to oligomerize unprotected mononucleotides with cyanogen bromide (BrCN) and a divalent metal salt (MgCl₂ or CaCl₂) in an aqueous solution. The effectiveness of the template directed oligomerization is improved by the circular form of the DNA template to allow triplex directed oligomerization having the mononucleotides as the central strand. Further modification of this stable DNA template enhance template pre-organization and reaction efficiency. Such modifications include the use of attached primers that will not be covalently incorporated into the oligomer being synthesized. Large scale ON synthesis (several kilogram/day) can be realized because the circular DNA template appears stable to the reaction conditions. Multiple cycles of oligomerization reactions may be performed using the same template for catalytic template use as a catalytic template.

Relative to a single strand template directed ligation reaction, a triplex directing template greatly improves the association of the mononucleotide substrates to the template through both Watson-Crick and Hoogsteen hydrogen bonding. The improved template association allows for higher reaction temperatures which improves the kinetics of the oligomerization reaction and results in a highly efficient and faster synthetic method. Recycling of any nucleotide starting materials will be facilitated by the hydrolytic reversibility of phosphate activation with BrCN. The “green” aspects of this process include the minimal reagent requirement (BrCN only), the hydrolytic breakdown of BrCN to NaBr (Na from the nucleotide salt components), HBr, CO₂ and NH₃ as the only waste byproducts, and the aqueous based nature of the chemistry. In total, this “green” process could be revolutionary as a commercial method for large scale ON synthesis in terms of cost, efficiency and environmental impact.

Unlike current existing methods of DNA synthesis, there is no need for toxic organic solvents. There is also no need for pretreated nucleotides. Protecting groups and capping reactions are unnecessary. The aqueous environment and relatively harmless chemicals offers a significant improvement over the existing methods of non-enzymatic oligonucleotide synthesis that results in large quantities of toxic pollutants and requires significantly modified nucleotides.

The circular polynucleotide template also overcomes the difficulties of other nucleotide templates attributable to the stacking forces. By using both Watson-Crick and Hoogsteen hydrogen bonding, the base specific hydrogen bonds dominate the weaker stacking forces in the ordering of the nucleotides. It is the additional hydrogen bonds facilitated by the DNA triplex formation that allows the present invention to succeed in oligonucleotide synthesis where previous templates have failed. Those skilled in the art will appreciate the significant advantages provided by the organic, non-enzymatic, synthesis of oligonucleotides and an aqueous solution that results in relatively harmless bi-products.

It is therefore an object of the present invention to provide an environmentally sound method of synthesizing oligonucleotides.

It is another object of the present invention to provide a relatively inexpensive method of producing relatively large quantities of oligonucleotides.

### BRIEF DESCRIPTION OF THE DRAWINGS

FIG. 1 shows a schematic diagram illustrating Watson-Cric and Hoogsteen hydrogen bonds.

FIG. 2 is a schematic representation of a ligation reaction between two very short oligonucleotides.

FIG. 3 is a schematic diagram of the ligation experiment utilized to demonstrate the efficiency of a circular DNA template.

FIG. 4 is a schematic diagram of a double ligation reaction between three oligomers on a circular template.

FIG. 5 is a diagram of PAGE (polyacrylamide gel electrophoresis) demonstrating the efficiency of a ligation reaction using the present invention.

FIG. 6 is a schematic diagram illustrating repetitive use of the invention for DNA oligomerization.

FIG. 7 is a schematic diagram of an alternative embodiment of the invention having covalently attached primers.

FIG. 8 is a schematic representation of a linker unit utilized in an alternative embodiment of the invention.

FIG. 9 is a schematic diagram of two alternative embodiments of the invention and grafts illustrating their stability.

### DETAILED DESCRIPTION OF THE INVENTION

The present invention provides an approach for improving the thermodynamics of substrate binding to a DNA template by maximizing hydrogen bonding interactions. A pyrimidine-rich DNA template which binds to reacting purine nucleotides through both Watson-Crick and Hoogsteen hydrogen bonding results in a triplex structure with the reacting purine bound as the central strand of the triplex. Further improvement in binding and sequence specificity for purine-rich single strand DNA is due to the circular structure of the pyrimidine-rich strands of the triplex. Additional components may be introduced to further enhance substrate association and regiocontrol of template binding. Regiocontrol will be enhanced through incorporation of modified cytidine (C) derivatives to one side, the Hoogsteen side, of the circular DNA template. Cytidine protonation is required for Hoogsteen binding in the C⁺GC triplet. These C-derivatives [i.e., 5-methylcytidine (^{Me}C) or pseudoisocytidine (^{p1}C) and other derivatives] control which side of the circular DNA template binds in the Hoogsteen mode by controlling cytidine protonation by lowering the pKₐ (^{Me}C) or having a permanently protonated C-derivative (^{pi}C)]. Cooperative stacking and steric factors facilitate homogeneity in directional alignment of mononucleotides on the pre-organized template.

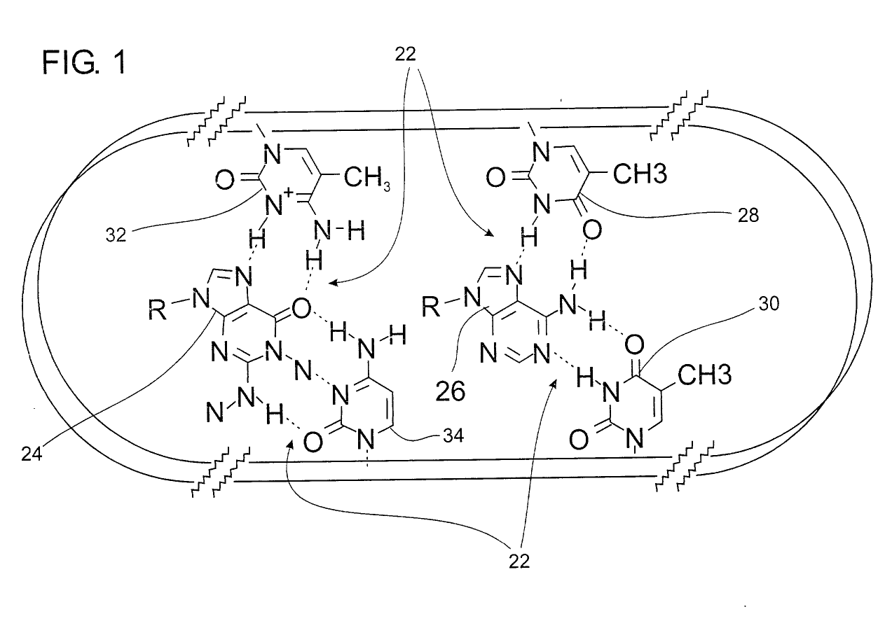

FIG. 1 is a diagrammatic illustration of hydrogen bonding between DNA base pairs. Standard Watson-Crickk base pairing 20 is illustrated between thymine (T) 30 and adenine (A) 26 and guanine (G) 24 and cytosine (C) 34. This is the base pairing found in “normal” DNA base pairing found in a regular double helix. Hoogsteen hydrogen bonding 22 is illustrated between methylated C 32 and G 24, and T 28 and A 26. When a purine base is subjected to both Watson-Crickk and Hoogsteen hydrogen bonding, the hydrogen bonding is almost doubled and thus the energy of the hydrogen bonding outweighs the base stacking forces. The present invention takes advantage of these added hydrogen bonds found in DNA triplex's. This allows the oligimerization of homopurines. The template is designed such that a methylated C and normal C pair are located where a G is desired in the oligimer. Similarly, a double T pair is located where an A is desired.

The present invention comprises a novel oligonucleotide synthesis template and method for using it. The template is comprised of two nucleotide regions, a Watson-Crick region and a Hoogsteen region. The Watson-Crick region is designed so that it will form normal, Watson-Crick hydrogen bonds with nucleotides in the desired sequence for the oligonucleotide to be synthesized. Similarly, the Hoogsteen nucleotide region is designed to form Hoogsteen bonds with nucleotides in the same sequence as the Watson-Crick region. These two regions are parallel to one another and are held together by at least one linker region. Preferably, there are two linker regions, one on each end of the parallel nucleotide regions. The linker regions may be comprised of nucleotides, polypeptides, or any of a variety of organic compounds. Those skilled in the art will appreciate that the purpose of the linker regions is to hold the two sequence encoding nucleotide regions close to one another so that a triplex may be formed with substrate nucleotides in the desired sequence.

The substrate may be either short oligonucleotides or individual mononucleotides. When the nucleotides are introduced to the template they form a triplex with the template in a base sequence determined by the sequences of the Hoogsteen and Watson-Crick nucleotide regions. A ligating reaction mixture is then added to the solution. This ligates the oligonucleotide that forms the central strand of the triplex.

To add stability to the template, it may be desirable to apply primers to the ends of the Watson-Crick and Hoogsteen nucleotide sequences. This adds stability and structure to the template. It also creates a partial triplex structure which facilitates and accelerates formation of the triplex structure with substrate nucleotides. Primers may be easily “capped” to prevent their ligation to the desired oligonucleotide. These primers may be covalently attached to the linker regions. This is another reason why it may be desirable to have linker regions comprised of molecules other than nucleotides.

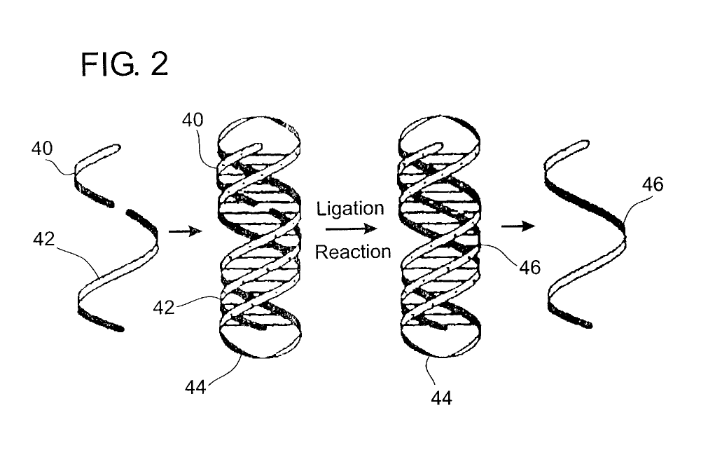

FIG. 2 is a diagramatic illustration of an initial “proof of concept” experiment performed to verify that the present invention was feasible. 6-MER 40 SEQ ID NO: 1 and 11-MER 42 SEQ ID NO: 2 were mixed with circular template 44 SEQ ID NO: 3 Template 44 SEQ ID NO: 3 was designed to bind oligimers 40 SEQ ID NO: 1 and 42 SEQ ID NO: 2 in a specific order. Because of the specificity of the Hoogsteen/Watson-Crick bonding, a triplex formed rapidly. The BRCN/metal salt polymerization reaction mixture was added to the mixture to bind the 2 short oligimers 40 SEQ ID NO: 1 and 42 SEQ ID NO: 2 in order to form 17-MER 46 SEQ ID NO: 4. Denaturation of the tri-plex loosens oligimer 46 SEQ ID NO: 4 from the template. This allows the template to be re-used in a subsequent reaction.

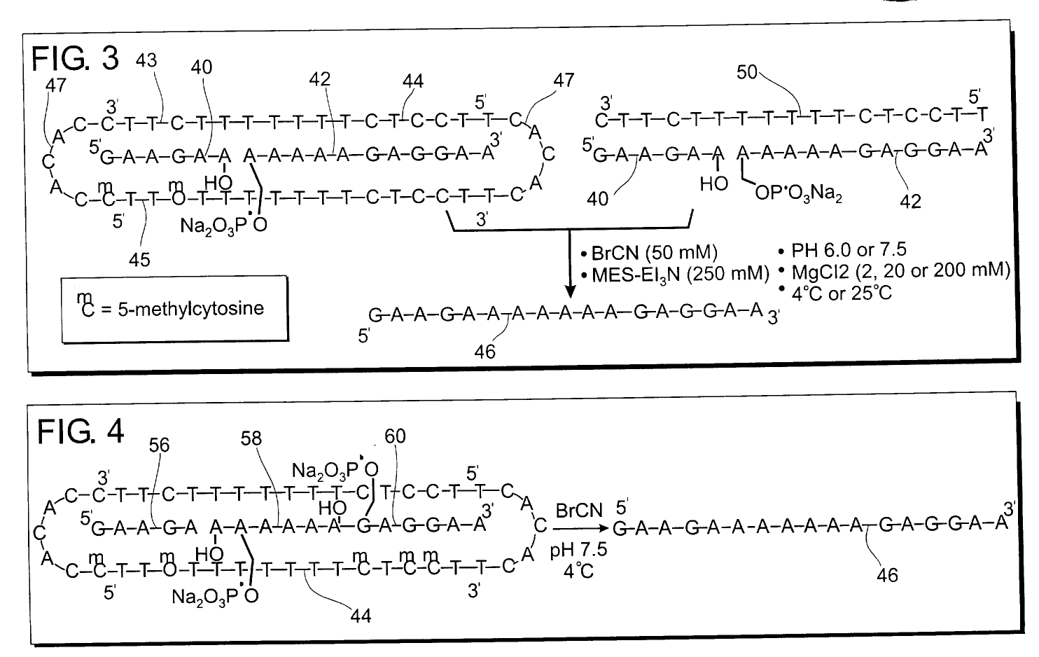

FIG. 3 illustrates the same “proof of concept” experiment of oligimer 40 SEQ ID NO: 1, of oligimer 42 SEQ ID NO: 2 and of template 44 SEQ ID NO: 3 and of oligimer 46 SEQ ID NO: 4 are all shown. Here, both the starting oligimers 40 SEQ ID NO: 1 and 42 SEQ ID NO: 2 and the end product 46 SEQ ID NO: 4 are homopurines. The base combinations illustrated in FIG. 1 are used to properly align oligimers 40 SEQ ID NO: 1 and 42 SEQ ID NO: 2 on template 44 SEQ ID NO: 3.

The BrCN activated chemistry for ligation and oligomerization of the template bound substrates was proven by this quantitative ligation yield of a short hexadeoxyribonucleotide (6-mer) with an 11-mer. The reaction is fast (nearly complete in <1 min) and can be accomplished under aqueous conditions from cheap, commercially available mononucleotides which are stable and require no protecting groups for the oligomerization and/or ligation process. The approximate 1,850-fold savings over conventional solid support approaches in material costs and the alleviation of waste concerns could revolutionize industrial large-scale synthesis of diagnostic and therapeutic homopurine oligonucleotides. The results obtained to date are unprecedented in the field in terms of yield and reaction conditions. Quantitative yields have been realized in ligation reactions with very short ON's (5-mers and 6-mers) on the circular DNA templates at 25 C, pH 7.5.

FIG. 3 compares oligimerization using a circular and a linear template which directs ligation through Watson-Crickk hydrogen bonding. In FIG. 3, template 50 SEQ ID NO: 5 is shown. It is designed to form standard Watson-Crickk hydrogen bonding with oligimers 40 SEQ ID NO: 1 and 42 SEQ ID NO: 2. The reaction mixture is then added to these oligonucleotides and oligimer 46 SEQ ID NO: 4 is then formed.

In FIG. 3, it can be seen that template 44 SEQ ID NO: 3 is comprised of a Watson-Crick nucleotide region 43 and a Hoogsteen nucleotide region 45. They are connected by two linkers 47 that are comprised of nucleotides.

Ligation directed by the circular DNA template was more efficient due to the improved binding affinities through both Watson-Crick and Hoogsteen hydrogen bonding to the ligating fragments. The effects of various parameters were studied in the cyanogen bromide (BrCN) activated ligation reaction including the substrate/template ratio, buffer, salt, ionic strength, pH and temperature. The optimal conditions for ligation on the linear template afforded 51% yield of ligated product 46 SEQ ID NO: 4 (pH 6.0, 200 mM MgCl₂, 4° C.). In contrast, near quantitative ligation on the circular template occurred at higher pH, higher temperature, and showed less dependence on Mg²⁺ concentration (97% yield, pH 7.5, 200 mM MgCl₂, 25° C.). The relative rate of the ligation reaction is approximately 23 times faster on the circular DNA template relative to the linear template (pH 7.5, 200 mM MgCl₂, 4° C.). These experiments show that chemical ligation of short ON's on circularized DNA templates through triplex formation is a highly efficient process over a broad range of conditions. This quantitative nonenzymatic ligation of two short ONs (6-mer+11-mer) is unprecedented and shows the tremendous potential for this method.

The template-substrate complex was analyzed with a combination of melting temperature (Tₘ) analysis, CD spectroscopy, and differential scanning calorimetry (DSC). These studies elucidated the nature of the template-substrate interactions. The circular template 44 SEQ ID NO: 3 binds the reacting ON's more tightly than single stranded template 50 SEQ ID NO: 5, effectively lowering the entropy of the ligation reaction through tighter pre-organization of the reacting ends (i.e., less fraying at the ends of the ON's on the template). The Tₘ analysis of the circular template with the two ligating substrates 40 SEQ ID NO: 1 and 42 SEQ ID NO: 2 compared to the single strand template 50 SEQ ID NO: 5 with 40 SEQ ID NO: 1 and 42 SEQ ID NO: 2 under the conditions for the ligation reactions confirms the tighter binding with the circular template. At 200 mM MgCl₂, both the circular template and the single strand template show melting above 25° C. (circular template-substrate complex: Tₘ=58° C.; single strand template-substrate complex: Tₘ=38° C.). However, only the circular template affords ligation product at 25° C. At 4° C., both templates should have the substrate ON's bound, yet the circular template 44 still reveals superior templating properties based on ligation efficiency (both yield and reaction rate). This may be a result of more fraying of the ON substrates on the single strand template, or perhaps better conformational positioning of the reacting ends on the circular template.

The conditions used in the initial single ligation reactions on a circular DNA template may be applied to a double ligation reaction. FIG. 4 shows The double ligation of 5-mer 56 SEQ ID NO: 6+6-mer 58 SEQ ID NO:7 +6-mer 60 SEQ ID NO: 8 afforded full length 17-mer oligonucleotide 46 SEQ ID NO: 4 in 24% yield. The nonenzymatic ligation of three oligonucleotides of such short length is unprecedented.

Conditions used for the previous double ligations are applied to triple ligations of trimers, quadruple ligations of dimers, and oligomerizations of monomers on circular DNA templates.

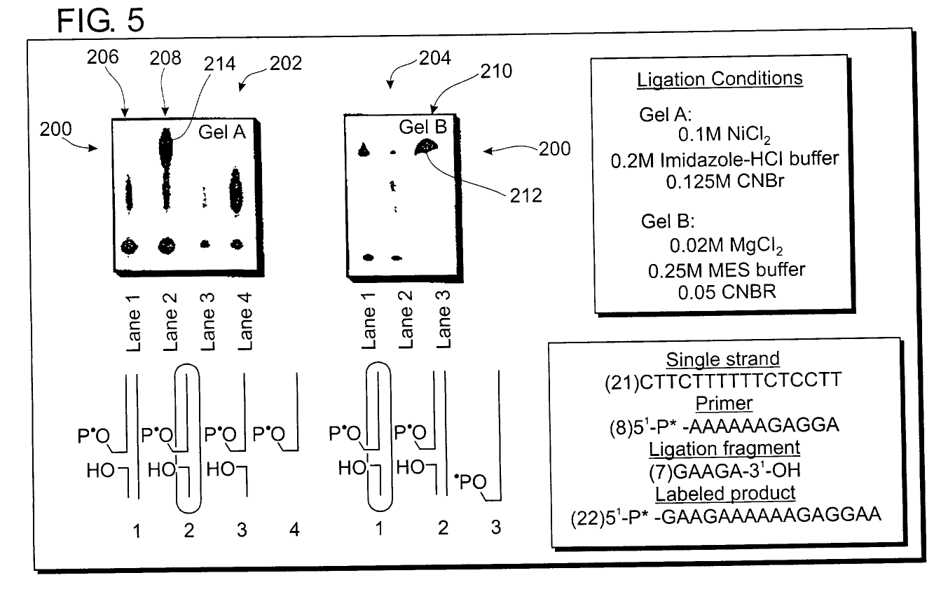

FIG. 5 illustrates a PAGE test done to show the effectiveness of a circular template. Gel 204 shows a band 212 in lane 210. It has migrated to point 200. This band represents the desired oligimer end product. In adjacent Gel 202, there is a similar band 214 in lane 202 that has migrated to point 200. Band 214 shows the results of an oligimerization reaction like the one shown in FIG. 2. Lane 206 shows the results of an oligimerization reaction done with a linear template. As can be seen, there is no band at point 200 in lane 206. FIG. 5 shows that using a circular template is a very successful method of oligmerization.

Ligation efficiency depends on the particular divalent metal ion used in the reaction mixture. Calcium (Ca(NO₃)₂) and magnesium (MgCl₂) are far superior in promoting the ligation reaction than any other divalent metal ion examined [including BaCl₂, MnCl₂, NiCl₂, CoCl₂, CuCl₂, ZnCl₂, and Fe(NH₄)₂(SO₄)₂.] The effectiveness of the metal ion is pH dependent and somewhat temperature dependent. Reaction efficiency is also dependent on the anion component of the metal salts. For example, There is a minor difference between (Ca(NO₃)₂) and CaCl₂.

Ionic strengths of 20-200 mM MgCl₂ are optimal. Ca(NO₃)₂ is equally as effective as MgCl₂ in the single ligation reactions. 0.5 M NaCl has minimal benefits on the double ligation reaction.

There is little difference in ligation efficiency based on whether the phosphate is on the 5′-end or the 3′-end of the ligating oligonucleotides.

Carbodiimide (EDCI) may also be used as the activating reagent for ligation Yields are approximately equivalent on the circular DNA template directed reactions, although the reactions are significantly slower.

The reaction mixture is comprised of a metal salt, BrCn, and a buffer to adjust the pH. Those skilled in the art will appreciate that the optimal pH and concentrations of chemicals will vary depending on temperature, size of the oligimer being synthesized, specific characteristics of the template, temperature and other factors known to those skilled in the art. The type of metal salt and buffer used will also depend on these characteristics.

Experiments have been performed involving ambient temperature ¹H NMR analysis of D₂₀ solutions of the dinucleotides (CpC and TpT) with BrCN at various concentrations. These experiments reveal no change to the dinucleotides which represent the key DNA components of the circular DNA template. The stability illustrated in this experiment means that the circular templates do not degrade. Because of this stability, the circular templates may be used repeatedly in several subsequent reactions. This not only speeds the oligimerization reactions, but further reduces the cost of forming oligimers using circular templates.

The C+GC triplet requires the Hoogsteen C to be protonated for two hydrogen bonds to be formed. This requirement establishes a handle for differentiating the two sides of the circular DNA template. The use of modified C derivatives on the Hoogsteen side of the circular template can enhance their potential for protonation. 5-methylcytidine (^{Me}C)³ can be replaced by modified derivatives which act as permanently protonated C derivatives, such as pseudoiso-cytidine (^{p1}C). Enforcing which side of the template will act as the Hoogsteen strand in the triplex complex by using modified C derivatives allows regioselective control (3′ vs. 5′ directionality) of substrate binding to the template. This minimizes pyrophosphate formation.

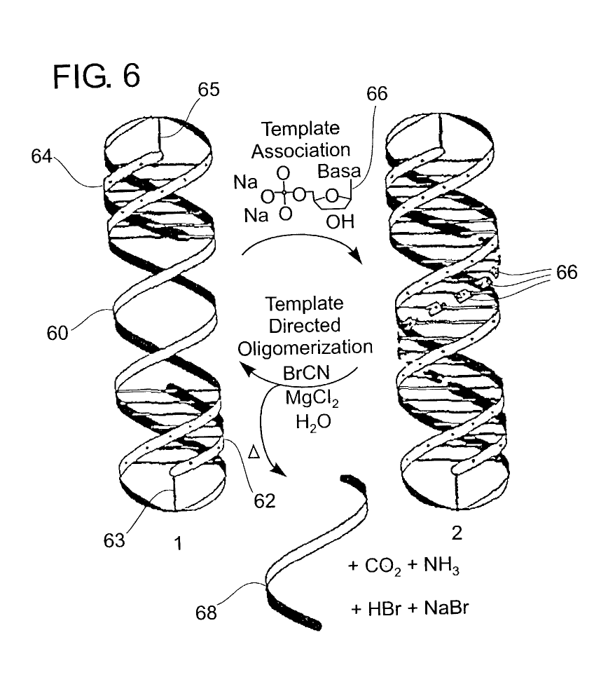

Prior to formation of the triplex, the circular template does not have a rigid structure, but rather is flexible. This slows the reaction time and the lack of stability can result in undesirable bi-products. To overcome these problems, primers may be added to the template. While the primers may simply be added to the reaction mixture, it is desirable to have the primers attached to either end of the templates as shown in FIGS. 6&7. Primer 64 is bound to template 60 by covalent bond 65. Similarly, primer 62 is covalently bound to template 60 by bond 63. By “capping” these primers on their unbound end, they are prevented from reacting with the desired oligonucleotide end product. The primers, like the desired oligonucleotide form both Watson-Crick and Hoogsteen bonds with the template, thereby initiating the triplex structure on either end of the template. This prepares the template for the oligimerization reaction by giving it a partial structure.

The incorporation of primers at each end of the circular template pre-organizes the template for substrate binding through triplex formation. These primers initiate regiocontrol by establishing which side of the template will bind in the Hoogsteen mode. Covalent attachment of these primers to the template affords a highly stable, pre-organized template for optimal substrate binding of mononucleotides or oligomers. The primers are capped in order to prevent their covalent incorporation into the ON being produced on the template. These aspects, combined with the template stability to the BrCN activated polymerization reaction conditions allows multiple cycles of template use. High turnover reaction methods allow the catalytic use of this template for large scale production of homopurine ON's.

FIG. 6 illustrates a preferred embodiment of a circular template. Template 60 has 2 primers 62 and 64 co-valently attached to templates 60. The attachment of these primers will be discussed in more detail below. Primer 62 and 64 add structure, stability and specificity to the template. Nucleotide monomers 66 are added to templates 60 to which they bond according the ace pairing described in FIG. 1. The reaction mixture is added to the solution. The monomers covalently bind to one another and denaturation of the oligimer-template complex results in release of oligimer product 68. After oligimer 68 is detached and removed from the solution, template 60 may be re-used. Those skilled in the art will appreciate that there are a number of methods of removing oligimer 68 from the template solution. Such methods include, but are not limited to, PAGE, centrifugation, chromatography and the dialysis method described above. This allows the synthesis and purification of shorter ODNs which can be combined to form larger and more complex template systems.

A computer program may be used to optimize the sequence of the primer and substrate binding regions of the circular template so that undesired hybridizations are minimized.

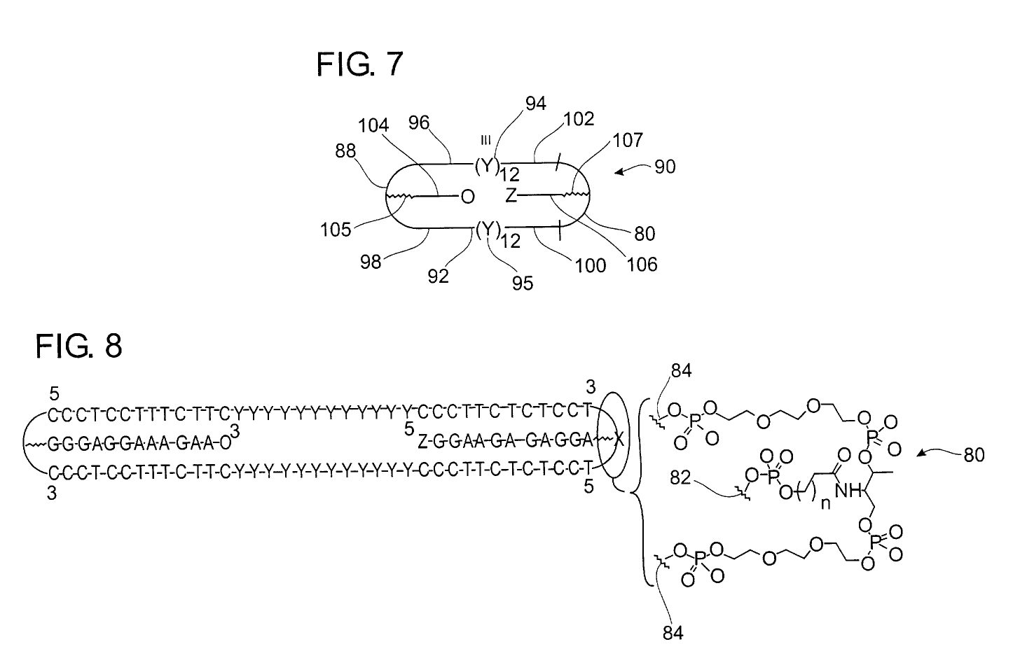

Those skilled in the art will appreciate that it may also be desirable to modify the ends of the circular template. FIG. 7 illustrates such a modified template. Circular template 90 has nucleotide sequences 102 and 100 that form a triplex with primer 106. Similarly, template 90 has nucleotide sequences 96 and 98 that form a triplex with primer 104. Template 90 has linker sequences 80 at either end. Linker sequences 80 are covalently bound to nucleotide sequences 96, 98 100 and 102. Linker sequence 88 is also bound to primer 104 by covalent bond 105. Similarly, linker 80 is covalently bound to primer 106 by covalent bond 107. Linker 80 may be a polypeptide or other organic compound. Those skilled in the art will appreciate that using a linker that is not comprised of polynucleotides can add stability and structure to the template. It may also facilitate synthesis of the template.

FIG. 8 shows a schematic diagram of an example of a linker. Linker 80 is an organic polyether compound. It has binding sites 84 capable of forming phosphodiaster bonds with nucleotides. Binding sites 84 are attached to template polynucleotides. Binding site 82 is designed such that it may bond with a primer. Those skilled in the art will appreciate that this is only one of many possible compounds that may be used for a linker.

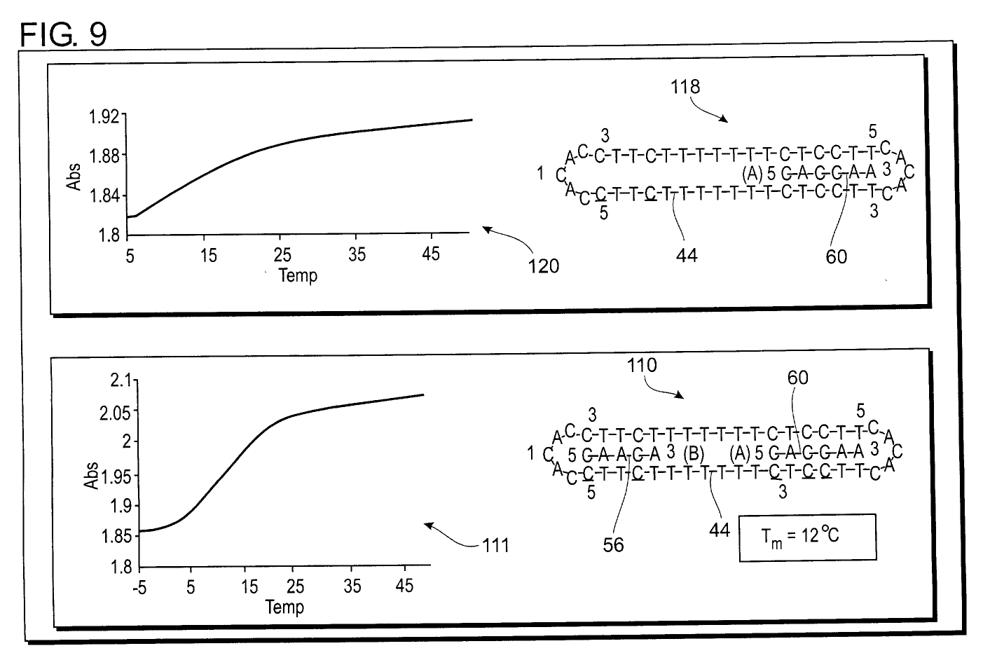

FIG. 9 shows the added stability provided by associating two primers with the template. Template complex 118 is comprised of circular template 44 SEQ ID NO: 3 and primer 60 SEQ ID NO: 8. As graph 120 illustrates, this templates denatures a too low of a temperature to accurately measure. Template complex 110 is comprised of circular template 44 SEQ ID NO: 3, primer 60 SEQ ID NO: 8 and primer 56 SEQ ID NO: 6. As graph 111 shows, this complex denatures at approximately 12° C. This shows that the use of two primers is more stable than using one. Using one primer, in turn, is more stable than using no primer.

The higher the number of substrates, the lower the percent yield. Using monomer substrates to form oligimers is the least efficient process. Therefore, the dialysis method described here consisting of subsequently longer and longer oligmerizations may be preferred.

The molecular weight (MW) of the smallest circular DNA template designed is approximately 18,000. This will be the lowest MW template used as all further template modifications will extensively increase the template size. The heptadecadeoxyribonucleotide product of ligation from this template has an approximate MW of 7,000. This MW difference is sufficient to allow separation with a 8,000 or 12,000 molecular weight cut off (MWCO) dialysis membrane. Advances in dialysis technology allows the use of a microdialysis system with dialysis snap-capped microtubes to avoid sample loss through the filling, tying and clamping of conventional dialysis tubing. Systems can be equipped for multiple sample capacity with oscillating and heating capabilities for efficient cyclical use. This allows for multiple cycles of template directed ON synthesis in a reaction vessel to which capped dialysis reservoirs containing the circular template are added. The substrates for template directed reactions are added to the reaction vessel, equilibrated for template association, The reaction is then initiated with addition of the reaction mixture After completion of the reaction the products are separated through a simple denaturation and washing sequence. One cycle of oligonucleotide synthesis consists of immersion of the dialysis reservoir containing the circular DNA template into a buffered reaction mixture containing the substrates to be ligated along with MgCl₂. Template-substrate equilibrium is established, then of BrCN is added to initiate ligation. After ligation (<30 sec), the solution is will be heated (for product denaturing), drained and washed (repeated as necessary). This cycle can be rapidly repeated (and readily automated) to produce the required amount of product.

For more efficient separation, the circular template can be modified through a PEG attachment. The biodegradable properties of this modification maintain the green aspects of this project. Solution based synthesis of oligonucleotides by either the phosphotriester method or the phosphoramidite method has been optimized by using a soluble PEG support. PEG-modified oligonucleotides are well know in the art.

The process engineering aspects of large scale ON synthesis using this technique may be accomplished in numerous ways. The most economic approach involves the sequential ligation reactions in a single reaction vessel with no purification in a type of in vitro selection process. Speed and simplicity are the primary advantages of this approach. Another approach involves sequential dialysis of each ligation reaction and separate reaction vessels for each consecutive ligation. High purity and reaction efficiency are the key advantages of this second approach.

In vitro Selective Ligations. This economically efficient approach requires only one reaction vessel for the synthesis of a given homopurine hexadecadeoxyribo-nucleotide. This is accomplished by having a reaction vessel containing all the required dinucleotides for ligation reactions to produce the four required tetranucleotides. Four separate MWCO 2,000 dialysis reservoirs, each containing a circular template designed for ligation of one of the tetramers, is added to the reaction vessel. These dialysis reservoirs allows the dinucleotide substrates to diffuse into the dialysis reservoirs and the tetramer ligation products of MW˜1,600 to diffuse out to the reaction vessel. A heating and cooling equilibration cycle allows template-substrate association. The low template association of mismatched dinucleotides and regioselective control imposed by primers and ^{Me}C or ^{p1}C incorporation in the template affords specificity in the template directed ligations. The ligation reaction is initiated by the addition of BrCN. After a brief reaction time (<1 min, to be optimized) the four dialysis reservoirs are removed, briefly washed, and any recovered tetramers added back to the reaction vessel. The dialysis reservoir containing the circular templates can be reused in separate reactions multiple times to afford the required amount of ON. The two circular templates required for tetranucleotide ligations to afford octanucleotides are added in MWCO 3,500 dialysis reservoirs to the reaction vessel. These dialysis reservoirs allow diffusion of the substrate tetranucleotides in and diffusion of the octanucleotide ligation products (MW˜3,200) back into the reaction vessel. The same template-substrate equilibration, BrCN ligation initiation, and brief wash affords the two desired octanucleotides in the single reaction vessel. Lastly, to this reaction vessel a dialysis reservoir (MWCO 8,000) containing the final template for octanucleotide ligation to afford the desired hexadecadeoxynucleotide is added. The same sequence as before affords a reaction mixture which should be highly concentrated with the final 16-mer. The buffered reaction conditions prevent significant pH changes as BrCN decomposition products build up over the course of the reactions. The dialysis reservoirs containing the circular templates can be reused as necessary to produce the required amount of ON. The final reaction mixture is concentrated in a MWCO 3,500 dialysis reservoir to allow concentration of the final 16-mer (MW˜6,400) from any shorter ON's in the solution (where the octamer will have a MW˜3,200). Any further purification, if required, can be accomplished by standard RP-HPLC or PAGE.

This allows a type of in vitro selection process for thermodynamic selection of the most favored template-substrate association in each step. If mismatched ligations occur at one stage, the products will associate less tightly with the template for the following ligation. Excess ON substrates from previous ligations similarly do not cause any complications since the longer ON ligation products will always associate more tightly with the circular template. The higher template association during ligation pre-equilibrium favors single ligation reactions of longer ONs to afford the intended product ON. The final dialysis will separate any starting substrates and truncated byproducts. This affords a superior, economical green process for homopurine ON synthesis for large scale therapeutic or diagnostic applications.

Sequential Dialysis Ligations. The second approach affords higher product purity from each ligation reaction, but requires more time. This approach involves sequential dialysis of each ligation reaction. Multiple reaction vessels are used, and a series of circular templates with attached primers in dialysis reservoirs are added. The initial ligation of two dinucleotides to afford the desired tetranucleotides is accomplished by adding the template in a MWCO 1,000 dialysis reservoir to a solution of the two required dinucleotides (MW˜800) followed by template-substrate equilibration for association. Ligation is then initiated with the addition of BrCN. After <1 min (conditions will be optimized), the dialysis reservoir with the product tetranucleotide (MW˜1,600) concentrated in the reservoir is removed, and transferred to a MWCO 2,000 dialysis reservoir. Heat denaturing dialysis affords the pure tetranucleotide. The circular template is then transferred back to the MWCO 1,000 dialysis reservoir for repeated use. This cycle can be repeated as necessary to produce the required amount of ligation product. The other three tetranucleotides are formed simultaneously in separate reaction vessels following the same procedure. The two desired tetranucleotides for the following octanucleotide synthesis combined in a reaction vessel to which the required template for the ligation reaction is added in a MWCO 2,000 dialysis reservoir (Step II, Scheme 14). Equilibration followed by ligation initiation with BrCN affords the product octanucleotide (MW˜3,200) concentrated in the dialysis reservoir. As before, heat denaturing dialysis from a MWCO 8,000 reservoir affords the pure octanucleotide. Again the remaining template can be reused for multiple ligation cycles to afford the desired amount of product. The same procedure is used to produce the additional required octanucleotide simultaneously. Following the same cycle, the two octanucleotides are placed in a reaction vessel to which a MWCO 3,500 dialysis reservoir containing the final circular template is added. Equilibration, BrCN initiated ligation, and denaturing dielysis from a MWCO 8,000 reservior affords the pure hexadecadeoxy-ribonucleotide. Repeated cycles afford as much product as required. This approach affords pure 16-mer homopurine ON's at any desired scale. The yield of each ligation is high, as is the final product purity since all precursors and by-products are removed at each stage of the sequential ligations.

Methodology Advantages. There are six important advantages to these approaches. (1) The catalytic use of the templates allows multiple cycles of each ligation reaction to be performed to produce large quantities of each oligonucleotide. (2) The high yield of each single ligation reaction allows high throughput and efficiency. (3) This dialysis based approach affords ON's of high purity and allows the circular templates to be efficiently reused with little or no loss of the template. (3) The use of unprotected nucleotides, cyanogen bromide and magnesium chloride in buffered water allows for a highly economic approach for the synthesis of defined oligonucleotides. (4) The fast rate of these ligation reactions also enhances the economics of this methodology through short cycle times. (5) This affords an optimal green process. The byproducts and waste from this synthetic approach are harmless to the environment. All unreacted ON's can be recycled to minimize the loss of any starting materials. (6) The combinatorialization of this methodology allows the synthesis of all possible homopurine hexadecadeoxyribonucleotides. The combination of all these advantages makes this methodology for homopurine ON synthesis an extremely attractive approach in regard to production scale, efficiency, cost, and environmental impact.

Synthesis of the PEG-modified circular DNA template may be accomplished through linear solution phase synthesis of a branched oligonucleotide on a PEG support followed by triplex directed circularization similar to that for which the template is used. However, difficulty in purification of the PEG-modified circular DNA product might make this approach less feasible. Postsynthetic modification of a circular DNA template with a PEG attachment through a non-nucleotide branch point in the template allows higher product purity. This can be accomplished by conventional automated, phosphoramidite chemistry with the inclusion of a non-nucleotide phosphoramidite having a Treoc-protected amino group for functionalization. Standard DMT-ON deprotection and cleavage from the solid support followed by purification with conventional RP-HPLC, removal of the DMT and a second RP-HPLC, will afford pure amino-protected, functionalized linear template. Triplex directed circularization by standard means followed by amino deprotection and PEG attachment through amidation will afford a PEG-modified circular template. Any underivatized circular template can be removed through dialysis.

Although the present invention has been described in terms of DNA, those skilled in the art will appreciate that this invention may be applied equally well to RNA. DNA was used for convenience in the illustrations and preferred embodiments. However, this is not intended to limit the invention only to DNA.

Whereas, the present invention has been described in relation to the drawings attached hereto, it should be understood that other and further modifications, apart from those shown or suggested herein, may be made within the spirit and scope of this invention.

What is claimed is:

1. A template for oligonucleotides synthesis comprising:
a Watson-Crick nucleotide region, having two ends;
a Hoogsteen nucleotide region, having two ends;
at least one linker region attaching at least one of said ends of said Watson-Crick nucleotide region and at least one of said ends of said Hoogsteen nucleotide region;
wherein, said Watson-Crick nucleotide region and said Hoogsteen nucleotide region are capable of forming a triplex with substrate nucleotides.

2. The template for oligonucleotide synthesis of claim 1, wherein said at least one linker region comprises two linker regions.

3. The template for oligonucleotide synthesis of claim 1, wherein said linker region is selected from the group consisting of an oligonucleotide, an oligopeptide, and a polyether.

4. The template for synthesis of oligonucleotides of claim 1 further comprising at least one primer.

5. The template for oligonucleotide synthesis claim 4, wherein said at least one primer comprises two primers.

6. The template for oligonucleotide synthesis of claim 4, wherein said at least one primer is covalently bound to said at least one linker region.

7. A method for synthesizing oligonucleotides comprising:
preparing a solution of substrate mononucleotides;
adding a circular polynucleotide template to said solution;
allowing said mononucleotide substrates and said circular polynucleotide template to form a triplex;
adding a reaction mixture to said solution, thereby causing the ligation of the mononucleotide substrates so as to form an oligonucleotide;
denaturing said triplex; and,
separating said oligonucleotide from said circular polynucleotide template.

8. The method of claim 7 further comprising adding a pH buffer to said solution.

9. The method of claim 7, wherein said reaction mixture comprises cyanogen bromide and a divalent metal salt.

10. The method of claim 9, wherein said divalent metal salt is selected from the group consisting of magnesium chloride, barium chloride, manganese chloride, nickel chloride, cobalt chloride, copper chloride, zinc chloride, calcium nitrate or calcium chloride.

11. The method of claim 9, wherein the concentration of said divalent metal salt is between 20 and 200 mM.

12. The method of claim 7 further comprising the step of increasing the temperature of said solution to greater than 10° C.

13. A method for synthesizing oligonucleotides comprising:
forming a solution of substrate nucleotides;
forming a solution of circular polynucleotide templates within a dialysis bag, wherein said dialysis bag allows difusion of olinucleotides but prevents difusion of circular templates;
immersing said dialysis bags in said solution of substrate nucleotides;
allowing triplex formation between said templates and said substrate nucleotides within said dialysis bags;
addition of the reaction mixture to said solution, thereby causing ligation of said substrate nucleotides to form an oligonucleotide;
denaturing said triplex, thereby dissociating said oligonucleotide from said template;
allowing said oligonucleotide to diffuse outside said dialysis bag; and
removing said dialysis bag from said solution.

14. The method of claim 13 further comprising raising the temperature of said substrate nucleotide solution to greater than 10° C.

15. The method of claim 13, wherein said substrate nucleotides is selected from the group consisting of mononucleotides, oligonucleotides, or polynucleotides.

16. The method of claim 13, wherein said reaction mixture is comprised of cyanogen bromide and a divalent metal salt.

17. The method of claim 16, wherein the concentration of said divalent metal salt is between 20 and 200 mM.

18. The method of claim 16, wherein said divalent metal salt is selected from the group consisting of magnesium chloride, barium chloride, manganese chloride, nickel chloride, cobalt chloride, copper chloride, zinc chloride, calcium nitrate or calcium chloride.


SEQUENCE LISTING (8 sequences)

SEQ ID NO: 1 - DNA, 6 bp, Artificial Sequence
  Misc_structure: oligonucleotide
gaagaa

SEQ ID NO: 2 - DNA, 11 bp, Artificial Sequence
  Misc_structure: Oligonucleotide
aaaaagaggaa

SEQ ID NO: 3 - DNA, 44 bp, Artificial Sequence
  Misc_structure 1, 3, 12, 14, 15: Template having 2′-O-methylcytidine at specified locations
cttctttttttctccttcacacttcctctttttttcttccacac

SEQ ID NO: 4 - DNA, 17 bp, Artificial Sequence
  Misc_structure: Oligonucleotide
gaagaaaaaaagaggaa

SEQ ID NO: 5 - DNA, 17 bp, Artificial Sequence
  Misc_structure: Oligonucleotide
cttctttttttctcctt

SEQ ID NO: 6 - DNA, 5 bp, Artificial Sequence
  Misc_structure: Oligonucleotide
gaaga

SEQ ID NO: 7 - DNA, 6 bp, Artificial Sequence
  Misc_structure: Oligonucleotide
aaaaaa

SEQ ID NO: 8 - DNA, 6 bp, Artificial Sequence
  Misc_structure: Oligonucleotide
gaggaa